(19) United States
(12) Patent Application Publication
(10) Pub. No.: US 2022/0076792 A1
(43) Pub. Date: Mar. 10, 2022

(54) METHODS FOR TREATING GROWING MEDIA CONTAINING PERSISTENT HERBICIDES

(71) Applicant: OMS Investments, Inc., Los Angeles, CA (US)

(72) Inventors: Tera Emilie Lewandowski, West Mansfield, OH (US); Nicholas Joseph Castorano, Powell, OH (US); Daisy Louise D'Angelo, Orient, OH (US); Kimberly Ann Rygielski, Powell, OH (US)

(21) Appl. No.: 17/469,128

(22) Filed: Sep. 8, 2021

Related U.S. Application Data
(60) Provisional application No. 63/075,675, filed on Sep. 8, 2020.

(51) Int. Cl. G16C 20/30; A01N 25/32; C05F 17/80

(52) U.S. Cl. CPC G16C 20/30; C05F 17/80; A01N 25/32

(57) ABSTRACT

A method of determining whether a treatment substance is effective for treating a growing media containing persistent herbicides is provided. The method may include determining two or more characteristics of the treatment substance and predicting a mitigation ability of the treatment substance to mitigate phytotoxicity caused by the persistent herbicides present in the growing media based on the two or more characteristics of the treatment substance.

### CROSS REFERENCE TO RELATED APPLICATIONS

This application claims the priority benefit of U.S. Provisional Patent Application No. 63/075,675, filed Sep. 8, 2020, which is hereby incorporated herein by reference in its entirety.

### TECHNICAL FIELD

The present disclosure relates generally to methods for treating growing media containing persistent herbicides and, more particularly, reducing or eliminating the detrimental effects of the persistent herbicides.

### BACKGROUND

Persistent herbicides are chemicals used to kill weeds or other unwanted growth that compete with desired plant growth, such as grass and grain crops. Examples include aminopyralid, clopyralid, aminocyclopyrachlor, and picloram. Often used to target weeds, persistent herbicides also damage some plants, such as broad-leaved plants (e.g., tomatoes, beans, etc.). Plant matter, such as grass clippings or hay, can be used in making compost material. When plant matter is treated with a persistent herbicide, it will then be present in resulting compost. Additionally, grass, hay, and grain are also used as animal feed, and the related manure and bedding are used to make compost material. If an animal's feed was treated with persistent herbicides, the persistent herbicide will likely be present in the manure or bedding. Generally, bacteria and/or fungi present in compost are able to break down herbicides and other problematic chemical compounds. However, persistent herbicides do not break down in the composting process and remain active in the finished compost material. When compost material containing persistent herbicides is used by consumers to grow plants, such as in gardens, the persistent herbicides can damage or kill the plants.

There is a need for improved treatments for material, such as compost, to mitigate the damaging residual effects of persistent herbicides.

### SUMMARY

In an embodiment, a method of determining whether a treatment substance is effective for treating a growing media containing persistent herbicides is provided. The method may include determining two or more characteristics of the treatment substance and predicting a mitigation ability of the treatment substance to mitigate phytotoxicity caused by the persistent herbicides present in the growing media based on the two or more characteristics of the treatment substance. The method may also include determining an amount of expected damage to a desired plant to be caused by the growing media once treated with the treatment substance. The two or more characteristics of the treatment substance may be selected from an adsorptive capacity, a density, a pH, or a manganese content. Four characteristics of the treatment substance may be determined and comprise an adsorptive capacity, a density, a pH, and a manganese content. The growing media may be, for example, compost. The treatment substance may be, for example, a carbon-based sorbent. The method may also include generating a predictive model for determining an acceptable treatment substance for treating the growing media containing the persistent herbicides. Predicting the mitigation ability of the treatment substance to mitigate phytotoxicity may include comparing the two or more characteristics to the predictive model. A coefficient of determination of the predictive model may be 75% or greater. The method may also include treating the growing media containing the persistent herbicides with the treatment substance.

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1A shows photographs of clover plants exposed to different compost samples that were analytically tested to contain approximately 30 ppb clopyralid.

FIG. 1B shows photographs of clover plants exposed to different compost samples that were analytically tested to contain no clopyralid.

FIG. 2 depicts a bioassay phytotoxicity scale of bio-injury to clover plants.

FIG. 3 is a regression analysis of percent carbon and the average clover damage for 28 different carbon-based sorbents.

FIG. 4 is a regression analysis of percent carbon and the average clover damage for 24 of the 28 different carbon-based sorbents of FIG. 3.

FIG. 5 is a regression analysis of adsorptive capacity and the average clover damage for the 24 different carbon-based sorbents of FIG. 4.

FIG. 6 is a regression analysis of density and the average clover damage for the 24 different carbon-based sorbents of FIG. 4.

FIG. 7 is a regression analysis of pH and the average clover damage for the 24 different carbon-based sorbents of FIG. 4.

FIG. 8 is a regression analysis of manganese content and the average clover damage for the 24 different carbon-based sorbents of FIG. 4.

FIG. 9 shows a main effects plot of the fitted means to show the magnitude and direction of the adsorption, density, pH, and manganese content in the fitted model.

### DETAILED DESCRIPTION

Various non-limiting embodiments of the present disclosure will now be described to provide an overall understanding of the principles of the function and use of the methods and processes disclosed herein. Those of ordinary skill in the art will understand that methods and processes specifically described herein are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features or characteristics may be combined in any suitable manner in one or more embodiments.

Some treatment substances, such as activated carbon, biochar, and wood ash (i.e., carbon-based sorbents), have been identified as potential remedies to phytotoxicity from persistent herbicides in compost material. Some of these treatment substances, such as biochar, may provide benefits unrelated to phytotoxicity. However, results using these treatment substances to remedy phytotoxicity were unpredictable. Although it had been suggested that carbon content was a predictive factor, testing with different sources of wood ash and biochar showed that not all sources work well, and that the carbon content of wood ash is not an accurate predictor of its ability to mitigate herbicide phytotoxicity, as discussed further in the examples below.

Compositions and methods for treating growing media containing persistent herbicides are described herein. The methods may be useful for mitigating the residual effects of persistent herbicides present in the target growing media (e.g., compost, soil, etc.). Growing media could include, for example, coir (compressed, non-compressed, screened, coir dust, and/or coir pith), peat, peat moss (for example, sphagnum peat moss), peat humus, vermiculite, compost, perlite, bark, bark fines, composted bark fines, wood shavings, sawdust, mulch, a modified cornstarch, corn stover, sunflower stem, composted rice hulls, reed sedge peat, composted manure, composted forest products, coffee grounds, composted paper fiber, digested manure fiber, composted tea leaves, bagasse, yard waste, cotton derivatives, vegetative by-products, agricultural by-products, or combinations thereof.

Various embodiments include a method of determining whether a treatment substance is acceptable for treating a target growing media containing persistent herbicides. Examples of treatment substances include, without limitation, a carbon-based sorbent, such as activated carbon, biochar, wood ash, etc. Suitable examples of biochar include materials that fall under the biochar definition from the Association of American Plant Food Control Officials (AAPFCO) or the International Biochar Initiative (IBI). Determining whether the treatment substance is acceptable may be based on physical or chemical characteristics of the treatment substance. In an embodiment, the physical characteristics of the treatment substance comprise one or more of the adsorptive capacity, the density, the pH, and the manganese content. In an embodiment, the physical characteristics of the treatment substance consist of the adsorptive capacity, the density, the pH, and the manganese content. As discussed further in the examples below, the combination of adsorption, density, pH, and manganese have shown surprising improvement in the prediction of mitigating persistent herbicide phytotoxicity. In an embodiment, the method may include determining one, two, or two or more characteristics of the treatment substance and predicting a mitigation ability of the treatment substance to mitigate phytotoxicity caused by the persistent herbicides present in the target growing media based on the characteristics of the treatment substance. In an embodiment, a target growing media containing persistent herbicides may be treated with an acceptable treatment substance, as discussed further below.

In some embodiments, a model is used to predict a mitigation ability of a treatment substance to mitigate phytotoxicity of persistent herbicide present in a target growing media. An embodiment includes generating the predictive model for determining an acceptable treatment substance for treating a target growing media containing persistent herbicides. A model capable of predicting the ability of a treatment substance to mitigate phytotoxicity caused by persistent herbicides present in a target growing media may be based on, for example, the adsorptive capacity, density, pH, and manganese (Mn) content of the treatment substance. The model can provide a more accurate prediction than either the prediction based on carbon content or the prediction based on adsorptive capacity alone. In various embodiments, the coefficient of determination (R² or R−sq) of the predictive model may be 75% or greater, 80% or greater, 85% or greater.

In various embodiments, a method may include determining whether a treatment substance is acceptable to treat a target growing media based on a threshold. For example, whether the treatment substance is acceptable may be determined based on a predetermined damage threshold of expected damage to a plant to be caused by the treated growing media. The predetermined damage threshold may vary based on the intended application. For example, the predetermined damage threshold may be zero damage. In an embodiment, the predetermined damage threshold may be based on a scale of damage, such as the bioassay phytotoxicity scale discussed in Example 2 (see FIG. 2). In some embodiments, the treatment substance may be determined to be acceptable if its adsorptive capacity, pH, density, manganese content, or a combination thereof would cause less damage than the predetermined damage threshold based on the predictive model.

The present disclosure can be further understood by reference to examples, of which summaries and detailed descriptions follow. These examples are provided by way of illustration and are not meant to be limiting.

### Example 1

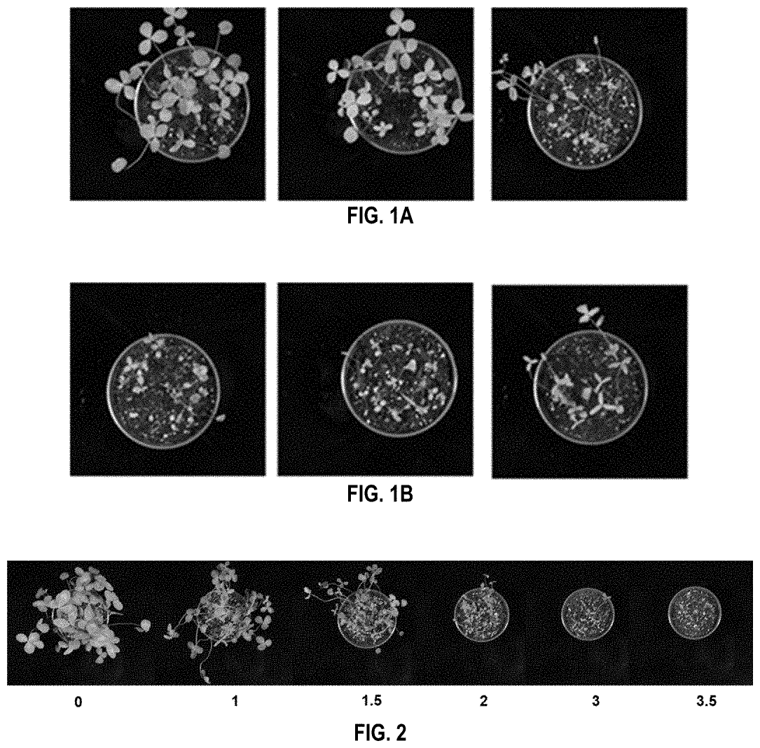

Quality monitoring was done through analytical lab testing to identify persistent herbicide concentrations in compost, and tests were conducted to determine if the analytically determined concentration correlated to expected effects of clover bio-injury. The results are shown in FIGS. 1A and 1B. In Experiment A (FIG. 1A), compost samples that were analytically tested to contain approximately 30 ppb clopyralid caused damage ranging from slight (left and middle) to severe (right). In Experiment B (FIG. 1B), compost samples that were analytically tested to contain no clopyralid all caused injury. Accordingly, clover bio-injury was observed during growth in clopyralid contaminated compost samples (Exp. A) and “uncontaminated” compost samples (Exp. B). The results showed that the analytical method of identifying the concentrations was ineffective.

### Example 2

Tests were conducted to evaluate the use of bioassays to determine the breadth and depth of the herbicide problem in composts. Initially, an internal clover bioassay phytotoxicity scale was established and validated, and the repeatability was demonstrated. Secondly, clover injury raters were trained based on a method developed at Woods End Research labs, as shown in FIG. 2. Using this sampling method, over 150 different compost samples were tested. Herbicide phytotoxicity symptoms were observed in 38% of the samples.

### Example 3

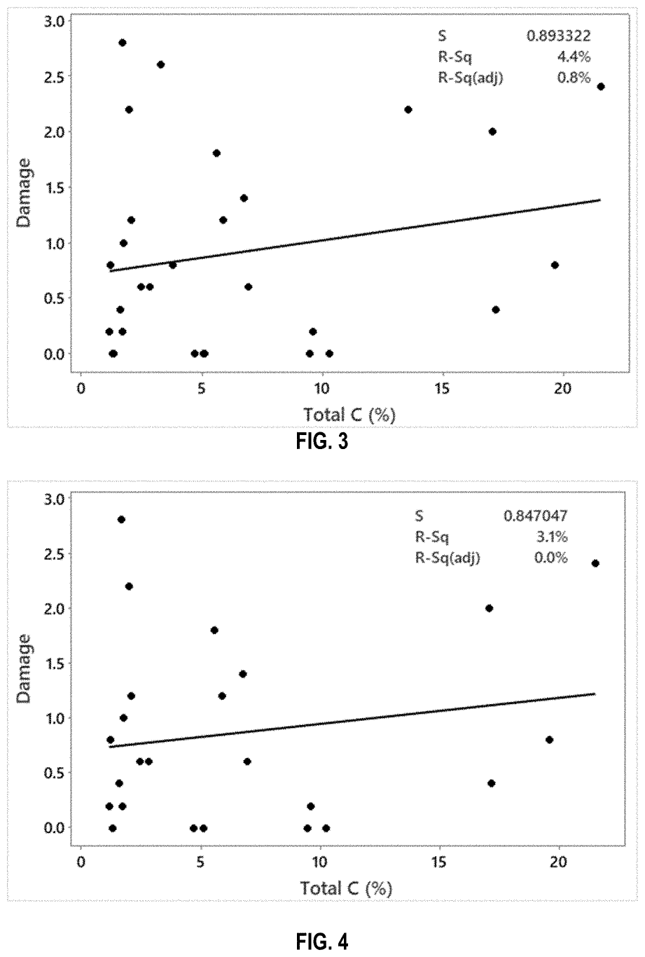

As discussed above, testing was conducted with different sources of wood ash and biochar to determine the effectiveness of mitigating herbicide phytotoxicity. Compost was spiked with 80 ppb clopyralid, then 5% by volume of 28 different carbon-based sorbents was added and performed clover bioassays on the blends. The results were analyzed to determine whether carbon content was predictive of the mitigation of the herbicide phytotoxicity. FIGS. 3 and 4, respectively, show a regression analysis of percent carbon and the average clover damage for 28 different carbon-based sorbents (R−sq=4.4%) and a reduced sample set of 24 different carbon-based sorbents (R−sq=3.1%), which is discussed further below. Some of the carbon-based sorbents worked well to alleviate the herbicide damage, while others had no effect. As shown, carbon content alone is not an accurate predictor of the ability to mitigate herbicide phytotoxicity.

The results were then analyzed to determine whether certain physical and chemical variables were predictive of the mitigation of the herbicide phytotoxicity. The variables are listed below in Table 1 along with their respective simple linear regression results.

TABLE 1

| Parameter | P-Value | R-sq | R-sq (adj) | R-sq (pred) |
|---|---|---|---|---|
| Adsorptive Capacity | 0.000 | 56.55% | 54.58% | 48.4% |
| Ash Weight % | 0.030 | 20.61% | 16.83% | 2.48% |
| Density | 0.849 | 0.17% | 0.00% | 0.00% |
| pH | 0.010 | 26.38% | 23.03% | 13.81% |
| Salts | 0.167 | 8.50% | 4.34% | 0.00% |
| Na | 0.117 | 10.80% | 6.75% | 0.00% |
| Ca | 0.705 | 0.67% | 0.00% | 0.00% |
| Mg | 0.015 | 24.89% | 21.32% | 11.73% |
| K | 0.179 | 8.04% | 3.87% | 0.00% |
| Zn | 0.908 | 0.07% | 0.00% | 0.00% |
| Mn | 0.003 | 35.15% | 32.06% | 22.57% |
| Cu | 0.262 | 5.96% | 1.48% | 0.00% |
| Fe | 0.708 | 0.68% | 0.00% | 0.00% |
| P | 0.693 | 0.72% | 0.00% | 0.00% |
| NO₃ | 0.030 | 19.67% | 16.01% | 7.48% |
| B | 0.723 | 0.61% | 0.00% | 0.00% |
| Cl | 0.215 | 6.90% | 2.67% | 0.00% |
| NH₄ | 0.446 | 2.66% | 0.00% | 0.00% |
| Total N | 0.667 | 0.86% | 0.00% | 0.00% |
| Total C | 0.409 | 3.12% | 0.00% | 0.00% |
| CN Ratio | 0.562 | 1.55% | 0.00% | 0.00% |
| OM | 0.710 | 0.64% | 0.00% | 0.00% |

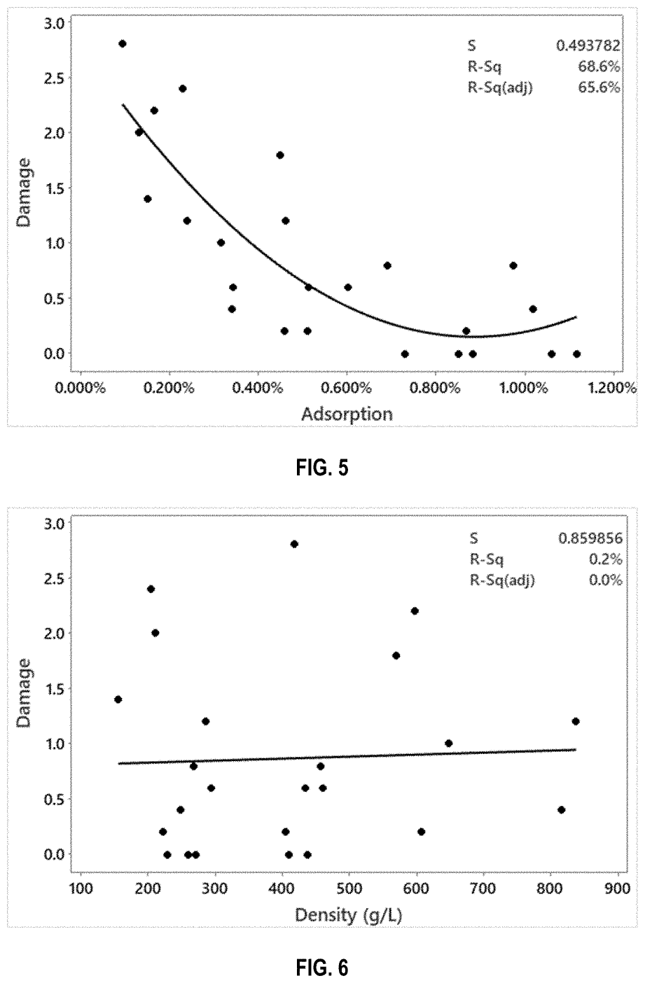

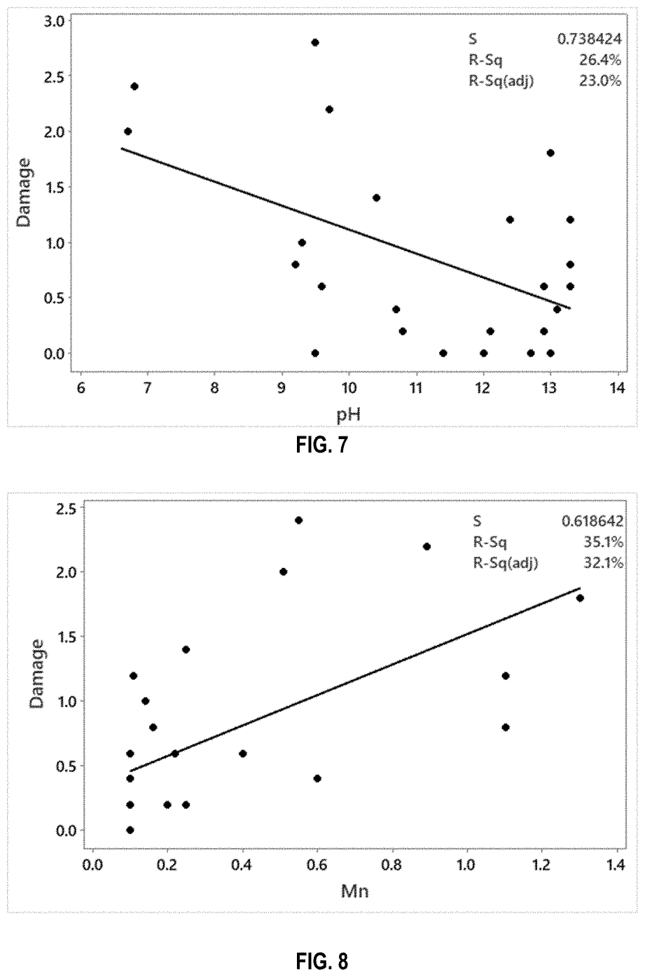

The adsorptive capacity of the 28 sorbents was measured using a Gravimetric Adsorption Capacity Scan (GACS) Assay, which is accomplished by challenging the sorbents with a known substance (e.g., an organic vapor) and then measuring the extent of uptake via adsorption of the challenge gas under controlled conditions. As shown in FIG. 5, compared to the carbon content (R−sq=3.1%), the adsorptive capacity (R−sq=68.6%) had a significantly higher predictive ability. FIGS. 6-8 show the regressions for density, pH, and manganese content of the different carbon-based sorbents. As shown in Table 1, the R−sq values for density, pH, and manganese content were about 0.2%, 26.4%, and 35.1%, respectively.

Surprisingly, a multiple regression analysis using the adsorption capacity, density, pH, and Mn content resulted in a highly significant model (adjusted R−sq=85%). The subset regressions are shown in Table 2.

TABLE 2

| | Ad + | Ad + | Ad + | Ad + | Density + |
|---|---|---|---|---|---|
| | Density + | Density + | Density + | pH + | pH + |
| Source | pH + Mn | pH | Mn | Mn | Mn |
| Regression | 0.000 | 0.000 | 0.000 | 0.000 | 0.000 |
| Adsorptive | 0.000 | 0.000 | 0.000 | 0.000 | — |
| Capacity | | | | | |
| (“Ad”) | | | | | |
| Density (g/L) | 0.068 | 0.725 | 0.005 | — | 0.890 |
| pH | 0.002 | 0.021 | — | 0.000 | 0.003 |
| Manganese | 0.000 | — | 0.000 | 0.000 | 0.001 |
| (Mn) | | | | | |
| R-sq | 87.86% | 70.19% | 78.84% | 85.31% | 61.53% |
| R-sq (adj) | 85.16% | 65.72% | 75.5% | 82.99% | 55.46% |
| R-sq (pred) | 80.34% | 58.23% | 69.29% | 79.04% | 46.51% |

The results of the combined model are shown in Table 3. Four of the data points were dropped during the model fitting procedure due to high residuals or outliers, leaving 24 data points in the final model. The variables were weighted equally.

TABLE 3
Analysis of Variance

| Source | DF | Adj SS | Adj MS | F-Value | P-Value |
|---|---|---|---|---|---|
| Regression | 4 | 10.8879 | 2.72198 | 32.55 | 0.000 |
| Adsorption | 1 | 3.2623 | 3.26231 | 39.01 | 0.000 |
| Density (g/L) | 1 | 0.3158 | 0.31580 | 3.78 | 0.068 |
| pH | 1 | 1.1175 | 1.11754 | 13.36 | 0.002 |
| Mn (ppm) | 1 | 2.4751 | 2.47507 | 29.60 | 0.000 |
| Error | 18 | 1.5051 | 0.08362 | | |
| Total | 22 | 12.3930 | | | |

Model Summary

| S | R-sq | R-sq (adj) | R-sq (pred) |
|---|---|---|---|
| 0.289169 | 87.86% | 85.16% | 80.34% |

In the full model, the correlation among predictors was checked and, as shown in Table 4, was low and not significant (p<0.05). In Table 4, the top value is the Pearson correlation and the bottom value is the P-value.

TABLE 4

| | Adsorption | Density (g/L) | pH |
|---|---|---|---|
| Density (g/L) | −0.338 | | |
| | 0.092 | | |
| pH | 0.279 | 0.227 | |
| | 0.168 | 0.265 | |
| Mn (ppm) | −0.240 | 0.308 | 0.101 |
| | 0.249 | 0.134 | 0.630 |

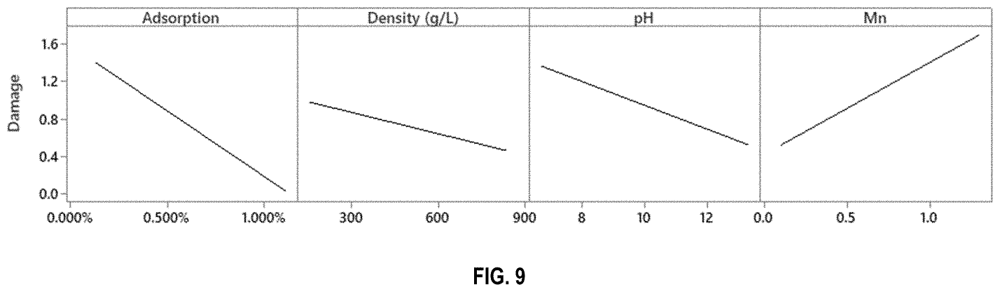

FIG. 9 shows a main effects plot of the fitted means to show the magnitude and direction of the significant predictors in the fitted model.

As used herein, all percentages (%) are percent by weight of the total composition, also expressed as weight/weight %, % (w/w), w/w, w/w % or simply %, unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. A method of determining whether a treatment substance is effective for treating a growing media containing persistent herbicides, the method comprising:
determining two or more characteristics of the treatment substance; and
predicting a mitigation ability of the treatment substance to mitigate phytotoxicity caused by the persistent herbicides present in the growing media based on the two or more characteristics of the treatment substance.

2. The method of claim 1, further comprising determining an amount of expected damage to a desired plant to be caused by the growing media once treated with the treatment substance.

3. The method of claim 2, further comprising comparing the expected damage to a predetermined damage threshold.

4. The method of claim 1, wherein the two or more characteristics of the treatment substance are selected from an adsorptive capacity, a density, a pH, or a manganese content.

5. The method of claim 1, wherein four characteristics of the treatment substance are determined and comprise an adsorptive capacity, a density, a pH, and a manganese content.

6. The method of claim 1, wherein the growing media is compost.

7. The method of claim 1, wherein the treatment substance is a carbon-based sorbent.

8. The method of claim 1, further comprising generating a predictive model for determining an acceptable treatment substance for treating the growing media containing the persistent herbicides.

9. The method of claim 8, wherein predicting the mitigation ability of the treatment substance to mitigate phytotoxicity comprises comparing the two or more characteristics to the predictive model.

10. The method of claim 8, wherein a coefficient of determination of the predictive model is 75% or greater.

11. The method of claim 8, wherein a coefficient of determination of the predictive model is 85% or greater.

12. The method of claim 1, further comprising treating the growing media containing the persistent herbicides with the treatment substance.
